(19) United States
(12) Patent Application Publication
(10) Pub. No.: US 2021/0000554 A1
(43) Pub. Date: Jan. 7, 2021

(54) Auto Home Zone and Slow Correction for Robotic Surgical System User Interface

(71) Applicant: TransEnterix Surgical, Inc., Morrisville, NC (US)

(72) Inventors: Matthew R. Penny, Holly Springs, NC (US); Taylor Fisher, Raleigh, NC (US)

(21) Appl. No.: 16/931,428

(22) Filed: Jul. 16, 2020

Related U.S. Application Data
(60) Provisional application No. 62/874,955, filed on Jul. 16, 2019.

(51) Int. Cl. A61B 34/00; A61B 34/30

(52) U.S. Cl. CPC A61B 34/25; A61B 34/30; A61B 2017/00199; A61B 2034/302; A61B 34/76

(57) ABSTRACT

A robotic surgical system in which a user input device may be dynamically adjusted to maintain beneficial ergonomic positioning, to maintain the input device in an optimal working range. The system includes a user input device having a range of motion in at least three degrees of freedom. Actuators may be operated to move the user input to a predetermined home position in response to user instructions, or to move the input device to a position in which the position of a portion of the input device in at least one of the degrees of freedom is in an intermediate portion of the corresponding range of motion.

This application claims the benefit of U.S. Provisional Application No. 62/874,955, filed Jul. 16, 2019.

### BACKGROUND

Surgical robotic systems are typically comprised of one or more robotic manipulators and a user interface. The robotic manipulators carry surgical instruments or devices used for the surgical procedure. A typical user interface includes input devices, or handles, manually moveable by the surgeon to control movement of the surgical instruments carried by the robotic manipulators. The surgeon uses the interface to provide inputs into the system and the system processes that information to develop output commands for the robotic manipulator. The user interface is designed to enable a more ergonomic positioning of the user's hands and arms. This means that the position and orientation of the user's hands and arms is no longer deterministic to the position of the surgical instrument end effector. In breaking this link between end effector and user interface, the surgeon can position the handles in an orientation that is more comfortable for the surgeon compared with the instrument handle positions during manual laparoscopic surgery. This helps to minimize the physical fatigue often associated with laparoscopic procedures. The user can maximize the ergonomics of the interface by “clutching,” which means temporarily disabling output motion at the surgical instrument in response to movement of the input device, to allow the surgeon to move the input device to a position that allows the surgeon to more comfortably manipulate the handle.

Another feature of physically separating the handle from the surgical instrument's end effector is that motion scaling is possible. This means that the user can adjust the relative amount of motion between the input and the output. If the user would like to create more precise motions at the instrument end effector, s/he can scale the end effector motion relative to the handle motion such that greater handle motion is required per unit of end effector motion. In this scenario, however, the user interface may have range of motion limitations where laparoscopic instruments did not. Once the surgeon has reached a range of motion limitation, s/he must “clutch out” in order to reposition the user interface prior to “clutching in” and regaining control of the instrument end effector.

Some systems are configured to communicate to the surgeon the forces that are being applied to the patient by the surgical devices moved by the robotic manipulators. Communication of information representing such forces to the surgeon via the surgeon interface is referred to as “tactile feedback” or “haptic feedback.” In systems such as the one described in application US 2013/0012930, tactile feedback is communicated to the surgeon in the form of forces applied by motors to the surgeon interface, so that as the surgeon moves the handles of the surgeon interface, s/he feels resistance against movement representing the direction and magnitude of forces experienced by the robotically controlled surgical device. In some systems, motors at the surgeon interface are also used to perform active gravity compensation at the user input devices.

This application describes a system and method that assists the surgeon in positioning of the user input device to maximize its range of motion, thus minimizing the impact and frustration of reaching range of motion limitations during use. It does so by controlling motors at the surgeon interface, such as those used to generate haptic feedback, to apply forces to the user input to cause movement of the user input to a predetermined position or region.

### BRIEF DESCRIPTION OF THE DRAWINGS

FIG. 1 shows an example of a robot-assisted surgical system;

FIG. 2 shows an example of a user input device for a robot-assisted surgical system;

FIG. 3 shows a embodiment in which a user input device is mounted to a moveable structure;

FIG. 4 schematically illustrates dynamic scaling factors relative to the user input device in the third embodiment;

FIG. 5 is a block diagram schematically illustrating operation of the system of the third embodiment.

### DETAILED DESCRIPTION

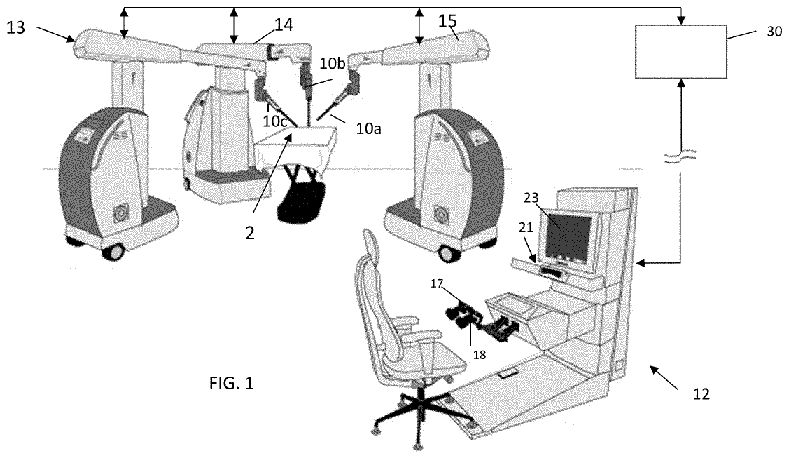

Although the inventions described herein may be used on a variety of robotic surgical systems, the embodiments will be described with reference to a system of the type shown in FIG. 1. In the illustrated system, a surgeon console 12 has two input devices such as handles 17, 18. The input devices 12 are configured to be manipulated by a user to generate signals that are used to command motion of a robotically controlled device in multiple degrees of freedom. In use, the user selectively assigns the two handles 17, 18 to two of the robotic manipulators 13, 14, 15, allowing surgeon control of two of the surgical instruments 10a, 10b, and 10c disposed at the working site at any given time. To control a third one of the instruments disposed at the working site, one of the two handles 17, 18 is operatively disengaged from one of the initial two instruments and then operatively paired with the third instrument. A fourth robotic manipulator, not shown in FIG. 1, may be optionally provided to support and maneuver an additional instrument.

One of the instruments 10a, 10b, 10c is a laparoscopic camera that captures images of the operative field in the body cavity. The camera may be moved by its corresponding robotic manipulator using input from a variety of types of input devices, including, without limitation, one of the handles 17, 18, additional controls on the console, a foot pedal, an eye tracker 21, voice controller, etc. The console may also include a display or monitor 23 configured to display the images captured by the camera, and for optionally displaying system information, patient information, etc.

A control unit 30 is operationally connected to the robotic arms and to the user interface. The control unit receives user input from the input devices corresponding to the desired movement of the surgical instruments, and the robotic arms are caused to manipulate the surgical instruments accordingly.

The input devices 17, 18 are configured to be manipulated by a user to generate signals that are processed by the system to generate instructions used to command motion of the manipulators in order to move the instruments in multiple degrees of freedom.

One or more of the degrees of freedom of the input devices are coupled with an electromechanical system capable of providing gravity compensation for the user input, and/or providing haptic feedback to the surgeon. It should be understood that the concepts described in this application are not limited to any particular user input device configuration. Alternative configurations include, without limitation, those described in co-pending application Ser. No. 16/513,670, entitled HAPTIC USER INTERFACE FOR ROBOTICALLY CONTROLLED SURGICAL INSTRUMENTS (Atty Ref: TRX-10610), and user interfaces or haptic devices known to those of skill in the art or developed in the future.

The surgical system allows the operating room staff to remove and replace surgical instruments carried by the robotic manipulator, based on the surgical need. Once instruments have been installed on the manipulators, the surgeon moves the input devices to provide inputs into the system, and the system processes that information to develop output commands for the robotic manipulator in order to move the instruments and, as appropriate, operate the instrument end effectors. The user interface may allow the surgeon to scale motion as well as to clutch and reposition the handles to a more comfortable position. In some cases, the surgeon may desire a fine scaling motion, while in others s/he may prefer larger motion, relative to the movement of the user interface.

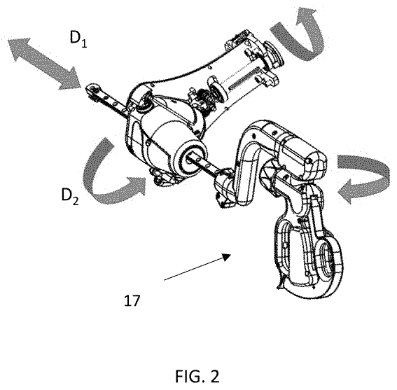

Each degree of freedom of the user input device has an associated range of motion. Purely by way of example, the input device shown in FIG. 2 may have, in the insertion degree of freedom D₁ (corresponding to the movement of the surgical instrument along it's longitudinal axis, or “insertion axis”) a travel range of approximately 170 mm, while the range of motion in the yaw degree of freedom D₂ might allow approximately 135 degrees between the limits.

### First Embodiment

The first embodiment allows the input device to be placed in a “home” zone or position that places the input device in a predetermined position along the range of motion/travel for each of its degrees of freedom. The position along each range of motion/travel may be predetermined for the system, or selectable by the user. In a preferred embodiment, the home position is one selected to optimize the range of motion/travel of the user input, thus reducing the likelihood that the input device will reach an end of its travel limits within a short period of time following initiation of use. By way of example, the starting position for the input device may be selected to be a position in the middle of the yaw and pitch range, and at the maximum extended insertion range, or at some other point of the insertion range.

Position sensors associated with the input device provide input to the system as to the real time position of the input device in each of its degrees of freedom. The system includes a processor coupled to a memory. The memory stores instructions executable by the processor to receive the input from the position sensors as well as input corresponding to instructions to place one or both of the input devices in the starting position, and to effect movement of the user input device(s) to the home position by causing the motors of the user input device(s) to reposition the user input device(s). This latter form of input may be given from any type of device that could deliver an input to the system. Non-limiting examples include an input control on the handle or elsewhere on the console or robotic system (as non-limiting examples, a switch, button, knob, key, lever, or touch input), a voice input device configured to receive verbal input, an eye tracker, head tracker, foot pedal etc. Alternatively, the processor may receive input from instrument engagement/detachment sensors at the robotic manipulator indicating when an instrument has been removed from a robotic manipulator, or when an instrument has been mounted to a robotic manipulator, either or both of which could service as input that triggers the system to place the user input device corresponding to the robotic arm undergoing the instrument attachment, detachment or exchange, in the starting position.

Once the input is received, the processor causes the motors of the user input device (e.g. the ones providing haptic feedback and/or gravity compensation) to automatically move the input device into a nominal starting position, which as discussed may be one that maximizes the range of motion in one or more directions. As a more specific example, the nominal starting position could be initialized whenever a new instrument is installed onto an arm and assigned to that specific user input.

### Second Embodiment

In a second embodiment, one or both of the input devices is automatically moved by the system in one or more degrees of freedom to place the user input device in a position and orientation to maximize the range of motion of the user input device, while minimizing the need for the surgeon to clutch to avoid reaching the limits of travel. This movement preferably occurs subtly during the time that the user is simultaneously manipulating the user input device to give input to the system for controlling a surgical instrument using the corresponding manipulator. The movement is actuated in response to detection by the system that the user input device is migrating in one or more degrees of freedom outside of a preferred 3-dimensional zone, and it is performed using actuators that may be independent from the actuators used for gravity compensation or tactile feedback.

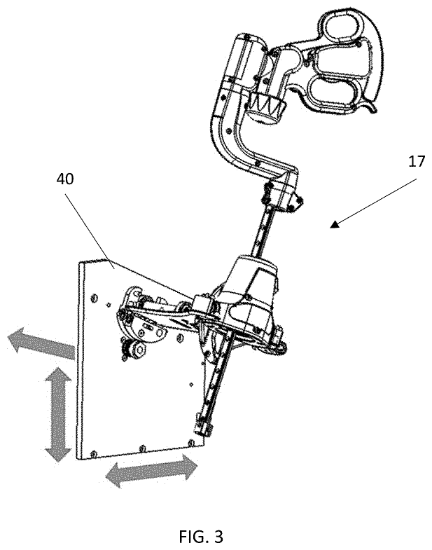

Referring to FIG. 3, in this embodiment, an input device 17 of the surgeon console is positioned on a support, which may be a frame or platform or other structure 40 that is capable of movement, but not dynamically involved in the generation of input for control of a robotic manipulator of the surgical system. In preferred embodiments, each input device 17, 18 is positioned on a platform 40 that is moveable relative to the surgeon console, preferably independently of the other platform. Actuators (not shown), which may be electromechanical, hydraulic, or pneumatic actuators, are positioned to move the platform. The platform 40 is moveable in at least one direction using the actuators to adjust the placement of the user input device 17 relative to the surgeon. It is optionally, but preferably, moveable in two or three directions, each of which is separately actuated (three orthogonal directions are indicated by arrows in FIG. 3).

The system includes a processor coupled to a memory. The surgeon console includes sensors associated with each degree of freedom of the user input device 17 and used to measure and monitor parameters such as mean position, movement duty cycle, and/or movement amplitude, etc. The system's memory includes data corresponding to three-dimensional positions and orientations of the user input device that maximize its range of motion in each degree of freedom. This data may correspond to, for example, a range of positions for the user input along each degree of freedom within which movement of the input device will optimize the ranges of motion of one or more of its degrees of freedom. The memory additionally stores instructions executable by the processor to receive input corresponding to the parameters, identify degrees of freedom that are being normally and regularly operated outside of the preferred ranges based on those parameters, and determine whether the position of the user input could be adjusted to bring that degree of freedom closer to the optimal, nominal positioning. If the processor determines that the position of the user input could be adjusted to do so, then the processor causes the actuator for that degree of freedom to move the platform so as to reposition the user interface to bring that degree of freedom into, or closer to, the optimal range. The rate of movement of the compensating movement of the platform in any or each degree of freedom may be selected so that the corrective movement of the user input by the platform is not, or is only nominally, perceptible to the user.

The system memory may include a single set of data setting the preferred range established for all users, multiple user-selectable preferred ranges (which may be categorized by task or procedure type or other characteristic of the anticipated motion of the user interface), or preferred ranges established for one or more specific users based on those users' previous history or preferences.

By using a platform or other mechanism to move the user input devices in one or more degrees of freedom (independent of their movement to give input signals to cause the robotic manipulator to manipulate the surgical instrument), the system places the user input device in a position and orientation that will maximize the range of motion of the user input device, while minimizing the need for the surgeon to clutch to avoid reaching the limits of travel.

### Third Embodiment

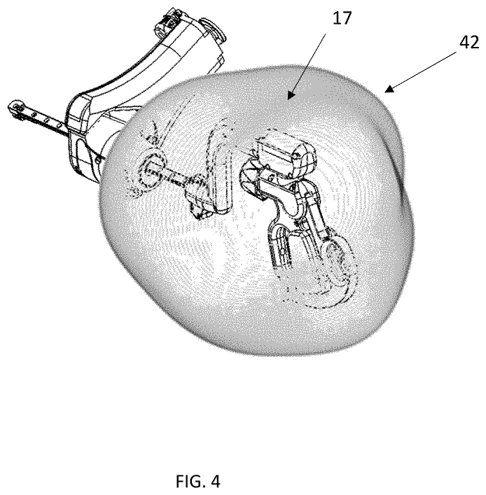

A third embodiment is similar to the second embodiment in that the system monitors different parameters such as those described above for each degree of freedom. However, this embodiment may be provided without a platform or other mechanism configured to move the user input devices in one or more degrees of freedom (independent of their movement to give input signals to cause the robotic manipulator to manipulate the surgical instrument). In this embodiment, if the processor determines based on input from the sensors that one or more degrees of freedom are being normally operated outside of the preferred range/zone, the processor dynamically adjusts the motion scaling relative to the degree of freedom imbalance. For example, if the user input device is consistently reporting a yaw movement between 80 and 120 on a scale of 0 to 135, the processor might dynamically adjust the motion scaling such that yaw movement towards 0 produces a slightly lower output than yaw movement towards 135. Theoretically, this would cause the surgeon to move the input further towards 0, than towards 135, which would correct the imbalance. As sensor input from all degrees of freedom is monitored simultaneously, it is anticipated that the dynamic scaling factor would have different amplitudes for different vectors of motion, and if plotted and overlaid with the input device, might look something like the amorphous cloud 42 depicted in FIG. 4.

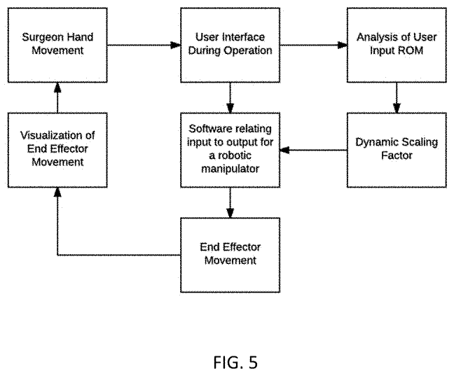

Thus, operation of the system using the dynamic scaling factor, combined with the anticipated user correction, would adjust the user input device into a more optimal range for all degrees of freedom. This would have the effect of minimizing clutching and optimizing surgeon ergonomics. FIG. 5 shows a flow chart detailing the system as it collects information from the user interface, calculates a dynamic scaling factor and feeds that back to the user input device.

The process flow chart of FIG. 5 illustrates that the system constantly analyzes the user input device movement at the user interface, adjusting a dynamic scaling factor and feeding that back into the software relating input to output for movement of the robotic manipulator carrying the surgical instrument. The end effector of the surgical instrument thus moves and the surgeon corrects his/her hand motion based on visualization of the end effector movement.

The concepts described in this application provide a number of advantages over existing technologies, particularly optimizing the range of motion of the user input device, and minimizing the number of clutches a surgeon must perform over the duration of a surgical intervention.

All patents and applications referred to herein, including for purposes of priority, are incorporated herein by reference.

1. A user interface for a robot-assisted surgical system, comprising:
a base;
a user input device moveable by a user in at least three degrees of freedom relative to the base;
sensors, each positioned to sense position of a portion of the input device in each of the degrees of freedom;
actuators operable to move the input device in each of the degrees of freedom; and
a processor directing operation of the actuators to cause movement of the input device in at least one of the degrees of freedom in response to user instructions directing placement of the user input device in a predetermined home position.

2. The user interface of claim 1, wherein the actuators are further operable to achieve gravity compensation.

3. The user interface of claim 1, wherein the actuators are further operable to deliver tactile feedback to a user moving the user input device, said tactile feedback represented forces between a surgical instrument and surrounding tissue or structures.

4. The user interface of claim 1, wherein the user instructions comprise signals generated by the system in response to mounting of an instrument to a robotic manipulator of the system.

5. The user interface of claim 1, wherein the user instructions comprise signals generated in response to user engagement with an input element.

6. The user interface of claim 1, wherein the user input device has a range of motion in each of said three degrees of freedom, and wherein in the home position the input device is positioned in an intermediate portion of the range of motion for at least one of the three degrees of freedom.

7. The user interface of claim 1, wherein the user input device has a range of motion in each of said three degrees of freedom, and wherein in the home position the input device is positioned in an intermediate portion of the range of motion for each of the three degrees of freedom.

8. A method of preparing a user interface of a robot-assisted surgical system, comprising:
providing a user input device moveable by a user in at least three degrees of freedom, and having a range of motion in each of said three degrees of freedom;
receiving user instructions directing placement of the user input device in a predetermined home position; and
in response to the user instructions, operating at least one actuator to cause movement of the input device in at least one of the degrees of freedom to place the input device in a home position.

9. The method of claim 8, wherein the user instructions comprise input generated in response to mounting of an instrument to a robotic manipulator of the system.

10. The method of claim 8, wherein the user instructions comprise input generated in response to user engagement with an auxiallary user input device.

11. The method of claim 10, wherein the auxiliary user input is selected from the group consisting of a member, knob, touch surface, acoustic sensor, optical sensor, eye tracker, body position sensor, head tracker, foot pedal.

12. The method of claim 8, wherein the method includes, in response to the user instructions, operating the actuators to cause movement of the input device in each of the degrees of freedom to place the input device in a home position.

13. A user interface for a robot-assisted surgical system, comprising:
a base;
a structure moveable on the base in at least one direction relative to the base;
a user input device on the structure, the user input device moveable by a user in at least three degrees of freedom relative to the structure, wherein the user input device has a range of motion in each of said three degrees of freedom
sensors, each positioned to sense position of a portion of the input device in each of the degrees of freedom relative to the corresponding range of motion;
at least one actuator operable to move the structure in said at least one direction; and
a processor directing operation of the at least one actuators to cause movement of the structure in said at least direction in response to sensed positions of the portion of the input device in at least one of the degrees of freedom relative to the corresponding range of motion.

14. The system of claim 13, wherein the processor directs operation of the at least one actuators to cause movement of the structure in said at least direction in response to sensed positions of the portion of the input device in each of the degrees of freedom relative to the corresponding range of motion.

15. The user interface of claim 13, wherein the structure is moveable on the base in at least three directions, wherein the actuators are operable to move the structure in said at least three directions, and wherein the a processor directions operation of the at least three actuators to cause movement of the structure in said at least three directions in response to sensed positions of the portion of the input device in each of the degrees of freedom relative to the corresponding range of motion

16. A method of dynamically adjusting a position of a user input device, comprising:
providing a user input device moveable by a user in at least three degrees of freedom, and having a range of motion in each of said three degrees of freedom, the user input device on a moveable structure;
sensing position of a portion of the input device in each of the degrees of freedom relative to the corresponding range of motion;
in response to the sensed position, operating at least one actuator to cause movement of the structure to move the input device to a position in which the position of a portion of the input device in at least one of the degrees of freedom is in an intermediate portion of the corresponding range of motion.

17. (canceled)
